SEQUENCE LISTING (2 sequences)

SEQ ID NO: 1 - DNA, 26 bp, Artificial
  Synthetic primer
cccggggatctaccatgagcccagaa

SEQ ID NO: 2 - DNA, 26 bp, Artificial
  Synthetic primer
gagctcagatctcggtgacgggcagg

(19) United States
(12) Patent Application Publication
(10) Pub. No.: US 2008/0184393 A1
(43) Pub. Date: Jul. 31, 2008

(54) Auto-Regulated Expression Of Bacterial Isopentenyltransferase Gene Promotes T-DNA Transformation In Soybean

(76) Inventors: Zhanyuan J. Zhang, Columbia, MO (US); Xinlu Chen, Columbia, MO (US); Henry T. Nguyen, Columbia, MO (US)

Correspondence Address: LATHROP & GAGE LC, 4845 PEARL EAST CIRCLE, SUITE 300, BOULDER, CO, 80301, US

(21) Appl. No.: 12/023,237

(22) Filed: Jan. 31, 2008

Related U.S. Application Data
(60) Provisional application No. 60/887,488, filed on Jan. 31, 2007.

(51) Int. Cl. C12N 15/82; C12N 15/11; A01H 5/00

(52) U.S. Cl. 800/279; 800/294; 800/298

(57) ABSTRACT

Agrobacterium-mediated plant transformation with a transgene of interest is accomplished with high frequency of transformation by using a specially constructed vector. The vector combines the transgene of interest with an autoregulating promoter, such as the P_{SAG12} promoter, that controls expression of an IPT coding region. The IPT coding region may be replaced by another ORF coding for the expression of a polypeptide affecting at least one plant cell cycle pathway selected from the group consisting of cytokinin, auxin, and sugar pathways.

### RELATED APPLICATION

This application claims priority to U.S. provisional patent application Ser. No. 60/887,488 filed on Jan. 31, 2007, which is hereby incorporated by reference.

### SEQUENCE LISTING

This application is accompanied by a sequence listing that accurately reproduces the sequences described herein.

### BACKGROUND

1. Field of the Invention

The present invention pertains to compositions and methods for transforming plants with polynucleic acids. More particularly, transformation is mediated by Agrobacterium tumefaciens that has been genetically altered to couple an autoregulating promoter with a gene affecting plant cell cycle pathways, for example, in cytokinin, auxin, and sugar pathways. Transformation frequency is significantly improved when the selection scheme in the transformation procedure is optimized using a negative selection techniques.

2. Description of the Related Art

Crop biotechnology at its frontier consistently faces new challenges and opportunities. Genomic technologies currently provide a large number of gene sequences from major crops and other organisms. Where previously the challenge lay in identifying genes for possible transformation, the challenge now increasingly resides in generating sufficient number of transformed plants at a minimal cost for successful expression of the available gene sequences.

It is problematic that current plant transformation technologies for most crop species result in relatively low transformation efficiencies. The present lack of a high-frequency transformation protocol presently constitutes a limiting factor to efforts that are directed towards exploring and improving crop genomes. These efforts include, for example, new variety development, biochemical pathway analysis, gene targeting at precise chromosomal location, and genome tailoring via homologous recombination, etc. These needs are recognized, for example, in the discussion of low frequency of success rates as provided previously (Nagy A., 2000, Genesis 26:99-109; Ow D W., 2002, Plant Mol Biol. 48:183-200; Hanin M, Paszkowski J., 2003, Curr Opin Plant Biol. 6:157-162).

The low frequency of genetic transformation has become a limiting factor in various fields as shown, for example, by assessments to determine whether certain experiments are feasible and/or a need for study timeline acceleration in certain fields or techniques that require gene delivery. These techniques may include RNAi, activation tags, enhancer traps, functional complementation of large DNA inserts of Bacterial Artificial Chromosome (BAC) (For a discussion of this problem, see Pereira A., 2000. Transgenic Res. 9:245-260).

Significant progress has recently been made in advancing Agrobacterium-mediated transformation in major crop species. Two major breakthroughs highlight these achievements. First is the development and deployment of a super-binary vector, which carries extra virB, virC, virD, virE, and virG genes of A. tumefaciens. Until now, the super-binary vector has provided the most efficient Agrobacterium-mediated T-DNA transfer in certain plant species.

The super-binary vector is a proprietary technology, and so is less accessible to the public. Even so, the super-binary vector has been used to transform a variety of crops. The super-binary vector has been used in rice (Hiei et al., 1997, Plant Mol. Biol. 35:205-218; and Komari T., 1990, Plant Cell Rep. 9: 303-306), in maize (Ishida et al., 1996, Nature Biotechnol. 14:745-750; Zhao et al., 1999, U.S. Pat. No. 5,981,840; Zhao et al., 2004, U.S. Pat. No. 6,822,144), and in sorghum (Zhao et al, 2000, Plant Mol Biol. 44:789-798). These successes have corrected a long-standing misconception that monocots are not transformable by Agrobacterium.

A second breakthrough is the use of antioxidants, which have been shown to enhance the T-DNA transfer in certain crop species. For example: dithiothreitol (DTT) is coupled with phenol-absorbing polyvinylpolypyrrolidone (PVP) to facilitate the transformation of grape (Vitis vinifera L.) (Perl et al., 1996, Nat Biotechnol. 14: 624-628).

DTT may be coupled with L-cysteine to facilitate the transformation of sugarcane (Saccharum officinarum L.) (Enriquez-Obregon et al., 1998, Planta 206:20-27). Ascorbic acid and L-cysteine may similarly benefit the transformation of rice (Enriquez-Obregon et al., 1999, Plant Cell Tiss. Organ. Cult. 59:159-168). L-cysteine may similarly benefit the transformation of maize (Zea mays) (Frame et al., 2002, Plant Physiol 129: 13-22; Vega et al., 2007, DOI 10.1007/s00299-007-0463-z) and soybean (Zeng et al., 2004, Plant Cell Rep. 22:478-482).

A combination of L-cysteine and DTT with or without Na-thiosulfate is useful in transformation of soybean (Olhoft et al., 2003. Planta 216:723-735; Paz et al., 2004, Euphytica 136:167-179). This use of antioxidants in both monocot and dicot transformation has made it possible to accomplish adequately efficient T-DNA transfer without using the “super-binary” vector.

A third breakthrough is the deployment of genes controlling plant cell cycles including the RepA from germini-virus. The use of RepA has drastically improved the transformation efficacy in both hybrid and inbred maize, for example, as reported before (Gordon-Kamm et al., 2002, Proc Natl Acad Sci USA 99:11975-11980).

Due to its economical values as a primary oil and protein crop in the United States and many parts of the world, soybean has been studied extensively in many aspects, including genetics, seed biochemistry and molecular biology, as well as genomics. To date, there are available from soybean more than 398,000 Expressed Sequence Tags (ESTs), a number of soybean BAC libraries with over 35-fold genome coverage, a well-developed composite map, and several mapping populations (Stacey et al., 2004, Plant Physiol 135:59-70).

Despite steady increase in the amount of publicly available soybean genomic data in the past decade or so, it remains difficult to utilize these available genomic resources and/or to explore other genome-wide engineering approaches, such as gene targeting, T-DNA tagging and transposon mutagenesis. This dilemma is due to, at least in part, the relatively low efficiency in soybean transformation. Efforts to improve soybean transformation have been hindered by the lack of a high-frequency Agrobacterium-mediated transformation that provides a high quality of transgene integration. Current techniques also suffer from relatively slow transgenic recovery, which particularly impedes rapid development of soybean or maize variety by genetic engineering.

“High quality” transgenic events are characterized by simple transgene inserts. This type of high quality transformation is sometimes essential to minimize transgene instability and the complexity in gene functional analysis that can be caused by multiple gene insertions. The converse, i.e., low quality or complex integration, is not uncommon among existing published transformation processes, for example, as reported by Somers and Makarevitch (2004, Curr Opin Biotechnol. 15:126-131).

It is presently necessary to make a new breakthrough in improving soybean transformation process with a high quality of integration events. The same truth holds in transformation of other crop species. The most advanced plant transformation technologies discussed above have enabled maximal transformation frequencies approximating 30% in rice, 41% in maize, and 16% in soybean.

Historically, soybean has been one of the most difficult plant species to be transformed by use of Agrobacterium tumefaciens. An early Agrobacterium-mediated transformation protocol used soybean cotyledonary nodes as explant tissues and kanamycin as a selectable marker (Hinchee et al, 1988, Bio/Technol. 6:915-922). It has proven very difficult for public research groups to reproduce those results.

One soybean transformation process has used the bar (bialaphos resistance) gene as a selectable marker, and the first time use of bar gene for other plant transformation is reported in Thompson et al. (1987, EMBO J 6:2519-2523). The bar system has enabled an average of 0.7% transformation efficiency in soybean (Zhang et al., Plant Cell Tissue Organ Cult. 56:37-46), and the protocol has been reproducible and has been further improved (Zhang et al., 2000, Plant Genetic Engineering: Toward the Third Millennium, A. D. Arenciba (Ed), Elsevier Science B. V. (Amsterdam), pp 88-94; Olhoft et al., 2001a, Plant Cell Rep 20:731-737; Olhoft et al., 2001b, Plant Cell Rep 20:706-711).

The herbicide glyphosate (Roundup® a trademark of Monsanto of St. Louis, Mo.) has been used for negative selection to confirm Agrobacterium-mediated soybean transformation, leading to approximately 1% recovery frequency (Clemente et al., 2000, Crop Sci 40: 797-803).

More recently, the antioxidants L-cysteine, DTT, and sodium thiosulfate have been shown to enhance Agrobacterium-mediated soybean transformation (Olhoft et al., 2001a, Plant Cell Rep 20:731-737; Olhoft et al., 2001b, Plant Cell Rep., 20:706-711). However, the addition of these antioxidants especially L-cysteine increases the percentage of “escapes” (non-transformed plants) due to the counter-selection effect of the antioxidants. This escape phenomenon causes reduced transgenic recovery from plant tissues that have actually been transformed.

To overcome this problem, the HPT II gene has been used as a selectable marker and hygromycin B as a selective agent for plant recovery (Olhoft et al., 2003, Planta 216:723-735). In addition, combinations of the antioxidants L-cysteine, DTT, and sodium thiosulfate have been used during the co-cultivation stage, which results in a drastic increase in Agrobacterium-mediated transformation of soybean, for example, to approximately 16% efficiency in the soybean genotype “Bert”. However, the outcome of transformation employing hygromycin selection may be soybean genotype-dependent. Despite intensive efforts in using this selection system to transform various soybean genotypes, only “Bert” has been transformed successfully so far (Zhang et al., unpublished report).

The dual problems of both high percent escapes and less optimal recovery frequency caused by the antioxidant L-cysteine present in the co-cultivation is solved by refining the glufosinate selection schemes, as reported previously (Zeng et al., 2004, Plant Cell Rep 22:478-482). This technique has been used to improve transformation efficiency at about 5.5% in public soybean genotypes “Williams 82,” “Maverick” and “Jack.” The same selection schemes work equally as well for all three of these genotypes. Under this selection condition, the co-cultivation medium is amended with L-cysteine, DTT and sodium thiosulfate at concentrations of 3.3 mM, 1 mM and 1 mM, respectively. Paz et al. (2004, Euphytica 136:167-179) took a similar approach in refining glufosinate selection and achieved about 1-3% transformation efficiency in a number of public soybean genotypes.

The isopentenyl transferase (IPT) gene from the T-DNA region of the non-disarmed Ti-plasmid of Agrobacterium tumefaciens has been previously reported (Strabala et al. (1989, Mol Gen Genet 216:388-394; Bonnard et al., 1989, Mol. Gen. Genet. 216:428-438). The IPT gene has been used in combinations with an Ac transposable element as a positive selectable marker to derive marker-free transgenic aspen plants (Populus sp.) (Ebinuma et al., 1997, Proc Natl Acad Sci USA 94:2117-2121). Recently, Molinier et al (2002) used a construct carrying IPT expression cassette driven by a constitutive CaMV35S promoter to transform sunflower (Helianthus annuus L.) via Agrobacterium tumefaciens, leading to 3-fold improved transformation efficiency. Endo et al (2002) used combined IPT/iaaM/H expression driven by constitutive CaMV35S to promoter tobacco transformation. However, no transformation has been reported using an autoregulated IPT expression system.

IPT is a first-committed enzyme that catalyzes de novo cytokinin biosynthesis, i.e. the addition of isopentenyl pyrophosphate to the N⁶ of 5′-AMP forms isopentenyl AMP (McGaw and Burch 1995, Plant Hormones: Physiology, Biochemistry and Molecular Biology, 2nd Ed. Dordrecht, The Netherlands, Kluwer Academic Publishers; p. 98-117; Chen C., 1997, Physiol Plant 101:665-673). Isopentenyl AMP is the precursor of all other cytokinins, of which the three most commonly detected and physiologically active forms in plants are isopentenyl adenine (IPA), zeatin (Z), and dihydrozeatin (Mok and Mok 2001, Annu Rev Plant Physiol Plant Mol Biol 52:89-118).

Conditional transgenic recovery from either tobacco (Nicotiana tabacum) or lettuce (Lactuca sativa) has been reported which overexpresses the IPT gene under the control of dexamethasone (Dex)-inducible system. This system is known as the DEX system and is described with more details by Aoyama and Chua (1997, Plant J 11:605-612; Kunkel et al., 1999, Nat Biotech 17:916-919). Accordingly, the IPT gene may be used as a positive selectable marker under conditional selection in the DEX-inducible system. In this system, only transformed cells that are capable of detoxifying DEX develop into a transgenic plant. The presence of cytokinin produced from the IPT gene in this selection strategy allows only transformed tissues to go through morphogenesis. Most transgenic plants posses a single copy of IPT transgene and are morphologically normal.

The IPT gene has also been placed under the control of the senescence-specific SAG12 promoter from Arabidopsis (P_{SAG12}-IPT), as reported by Lohman et al. (1994, Physiol Plant 92:322-328). This expression cassette has been used to confer abiotic stress tolerance in plants, causing significantly delayed developmental and post-harvest leaf senescence in mature heads of homozygous transgenic lettuce under stress conditions. U.S. Pat. No. 6,359,197 issued to Amasino et al. (1997) describes the SAG12 promoter from Arabidopsis, together with other senescence-specific promoters from other plants. The '197 patent describes how SAG12 homologues may be identified as additional senesence-specific promoters from other plants and how the SAG12-IPT construct may be inserted into Agrobacterium and used to transform Arabidopsis and Nicotiana tabacum (tobacco). This expression cassette has not been used in soybeans.

### SUMMARY

The present disclosure advances the art and overcomes the problems outlined above by providing methods and materials for high frequency transformation of plants. Some plant species, such as soybean, have been known to be recalcitrant to transformation. The improved method disclosed here, namely, Agrobacterium-mediated transformation by use of the Psag₁₂-IPT expression cassette, represents a new accomplishment in the area of plant transformation.

More specifically, the method for transforming a plant with a transgene of interest may comprise the steps of (a) constructing a vector that contains (i) an autoregulating promoter coupled with an open reading frame to control the expression thereof, (ii) the open reading frame (ORF) coding for the expression of a polypeptide affecting at least one plant cell cycle pathway selected from the group consisting of cytokinin, auxin, and sugar pathways, and (iii) the transgene of interest; (b) incorporating the vector into an Agrobacterium; and (c) using the Agrobacterium to transform a plant by insertion of the vector to transform the plant. The method may also include a step of confirming transformation of the plant by a negative selection technique.

In another aspect of the present disclosure, the improved transformation method may include the steps of (a) modifying a binary plant transformation vector for an Agrobacterium tumefaciens-mediated transformation to incorporate an expression cassette comprising a P_{SAG12} promoter coupled with an IPT coding region for control thereof and a transgene of interest to provide a modified binary vector for Agrobacterium tumifaciens; and (b) using this modified binary vector for Agrobacterium tumifaciens to transform said plant.

In yet another aspect, an expression cassette uses a promoter designated as P_{SAG12} that is operably coupled with an IPT coding region or an open reading frame. The sequence of the P_{SAG12} promoter may be found under GenBank Accession # U37336, and the sequence of IPT gene is from nucleotide 7864 to 8586 of GenBank Accession # NC_{—}002377. The gene construct may be introduced into the genome of many plants, most preferably, the genome of soybean, by Agrobacterium-mediated transformation. This procedure provides a high quality integration event characterized by a high percentage of simple inserts among transgenic events. There is also a fast transgenic recovery in recalcitrant crop soybean. Use of P_{SAG12}-IPT provides an improved transformation in recalcitrant crop soybean with an average of 2.5 to over 6-fold increase of transformation frequency than standard control in many soybean genotypes. The highest transformation frequencies achieved using P_{SAG12}-IPT are 14% in Mustang and 12% in Magellan, respectively. Advantageously, such enhanced transformation by use of P_{SAG12}-IPT does not compromise the high quality (single- or very low copy in the range of 2-4 copies per genome) transgene integration events in soybean.

Hence, this is the most efficient transformation system in soybean described to date. This protocol should better serve the need for genome-wide functional genomics studies in soybean as well as other genetic engineering efforts.

Although the P_{SAG12} promoter is the preferred promoter, other autoregulating promoters may be used in place of the P_{SAG12} promoter. In another aspect, although the IPT gene is described by way of example in this disclosure, the use of other open reading frames (ORFs) encoding proteins affecting plant cell cycle pathways, for example, in cytokinin, auxin, and/or sugar pathways, may also be used for purpose of this disclosure. The use of an autoregulating promoter coupled with an ORF of this nature is shown by the experiments described in the Examples.

The terms “transgene of interest” and “gene of interest” may be used interchangeably throughout this disclosure. For purpose of this disclosure, a “transgene of interest” may include a gene encoding a selectable marker such as the “bar” gene, or any other economically important genes. The term “economically important genes” may include, but are not limited to those genes that, when introduced into a host plant, confer upon the host plant a phenotype that enhances the growth, yield, or other economic value of the plant. Examples of such economically important genes may include, without limitation, raffinose synthase gene, polyunsaturated fatty acid desaturase genes such as FAD2, FAD3, FAD6, genes that confer resistance to nematodes, fungi, or other pathogens, or genes that confer drought resistance. See e.g., Li et al., 2007; Napier, J A, 2007. See also, U.S. patent application Ser. No. 11/835,328 entitled “LysM Receptor-Like Kinases To Improve Plant Defense Response Against Fungal Pathogens” describing a large number of plant genes that may confer upon a transgenic plant the capability to resist fungal infection; and U.S. Patent Application Ser. No. 60/943,321 entitled “Drought Responsive Genes In Plants And Methods Of Their Use” describing a large number of plant genes that may confer drought resistance to a host plant.

### BRIEF DESCRIPTION OF THE DRAWINGS

FIG. 1 is a map of a binary plant transformation vector that carries the Psag₁₂-IPT expression cassette in forward orientation relative to a bar gene expression cassette, which may be used in Agrobacterium-mediated transformation processes.

FIG. 2 shows in vitro culture responses of an improved transgenic recovery of soybean using the Psag₁₂-IPT gene expression cassette for enhancing transformation (right) than standard control (left).

FIG. 3 shows verification of transgene integration by using herbicide leaf-painting to confirm the functional expression of bar gene.

FIG. 4A and FIG. 4B show the result of genomic Southern blot analysis obtained from different events of soybean.

FIG. 5 shows stable inheritance of IPT transgene to the progeny by Southern analysis of progeny plants from a randomly chosen transgenic event.

### DETAILED DESCRIPTION

There will now be shown and described a method of modifying an Agrobacterium tumefaciens-mediated DNA delivery by incorporating an expression cassette comprising a P_{SAG12} promoter coupled with an IPT coding region to provide a modified DNA delivery method; and using the modified Agrobacterium tumifaciens-mediated DNA delivery to transform soybean. The working examples that follow each use preferred materials and methods to illustrate the practice of what are claimed. As such, the examples should not be applied in a manner that unduly limits what is claimed. Citations to technical articles identify by author name and parenthetically the publication year to identify citations that are more completely set forth in the References section below.

The prior art shows that soybean [Glycine max (L.) Merr. is one of the most recalcitrant plant species to be engineered using Agrobacterium tumefaciens. Despite recent progress, T-DNA transformation in soybean still much less efficient than in some other crop species. The instrumentalities disclosed below make possible a higher frequency transformation process in soybean that better meets the needs of genome-wide gene functional analysis and other bioengineering efforts.

In one aspect, materials and methods are provided for high-frequency Agrobacterium tumefaciens-mediated T-DNA transfer in soybean. An expression cassette is constructed with a bacterial isopentenyl transferase gene (IPT) under the control of an auto-regulating promoter (SAG12). Expression of the Psag₁₂-IPT cassette has been evaluated under various transformation and regeneration conditions, leading to an improved and high-frequency transformation. Transgene inserts of primary (T₀) transformants have been confirmed by Southern blot analysis in plant samples randomly chosen from a large number of recovered plants.

### EXAMPLES

The following examples are intended to provide illustrations of the application of the present disclosure. The following examples are not intended to completely define or otherwise limit the scope of the invention.

### Example 1

### Psag₁₂-IPT Vector Construction

The bar gene described by Thompson et al. (1987) was placed in a binary plant transformation vector designated herein as plasmid pZY101. The plasmid pZY101 was designed to carry an expression cassette of the bar gene and a multiple cloning site (MCS). To achieve this, a plasmid vector pCAMBIA3300 that contains the bar gene was purchased on commercial order from CAMBIA of Canberra, Australia. The bar open-reading-frame (ORF) was first amplified by polymerase chain reaction (PCR) from the vector using sense primer 5′-CCCGGGGATCTACCATGAGCCCAGAA-3′ [SEQ ID NO. 1] and antisense primer 5′-GAGCTCAGATCTCGGTGACGGGCAGG-3′ [SEQ ID NO. 2]. The PCR cycle parameters included a five minute hot start and one minute denature at 94° C., one minute annealing at 68° C., and one minute extension at 72° C. for 35 cycles, followed by a seven minute final hold at 72° C. This manipulation added Sma I and Sac I restriction sites to flank the bar ORF.

The resultant PCR-fragment was digested with the above enzymes and subcloned into the corresponding sites in between the CaMV35S promoter that is described by Odell et al. (1985) and the soybean vegetative storage protein gene (VSP) terminator of the vector pIBT210.1 described by Mason et al. (1993). The resultant vector received the designation pZY100. The vector pIBT210.1 also carried tobacco etch virus (TEV) translational enhancer as described by Carrington and Freed (1990) downstream of the 35S promoter. The vector pZY100 was then digested with Nco I and Sma I, blunt-ended with mung bean nuclease purchased on commercial order from Promega of Madison, Wis. and re-ligated to remove the Nco I, BamH I and Sma I sites simultaneously at a 5′ location upstream of the bar ORF. This new vector was designated pZY100.1.

The Sac I site downstream of the bar ORF was subsequently removed by blunt-ending with the mung bean nuclease and re-ligation. The resultant vector was termed pZY100.2. Subsequently, the bar gene expression cassette including the 35S promoter, bar ORF, and the VSP gene terminator was excised as a Hind III-EcoR I fragment and subcloned into the Hind III site within the multiple cloning region of the binary vector pPZP202. This new binary vector was designated pZY101 (or PTF101). This vector has previously been used to derive other vectors including pZY101.1 (or PTF101.1) and pZY102 (or PTF102), as reported before (Frame et al., 2002; Olhoft et al., 2003; Zeng et al., 2004; Paz et al., 2004).

The Psag₁₂-IPT expression cassette is described by Amasino et al. (1997) and was produced according to methods described therein. This expression cassette carries a SAG12 promoter that drives the bacterial isopentenyl transferase gene (IPT) coding sequence and the nopaline synthase gene (nos) terminator. The cassette, which was originally subcloned into the pUC18 vector by Amasino et al. (1997), was excised as a Spe I fragment and subsequently subcloned into the Xba I site of the multiple cloning site of the binary vector pZY101 in two opposite orientations. The resultant vectors are referred to as pMUIPT-F and pMUIPT-R, respectively.

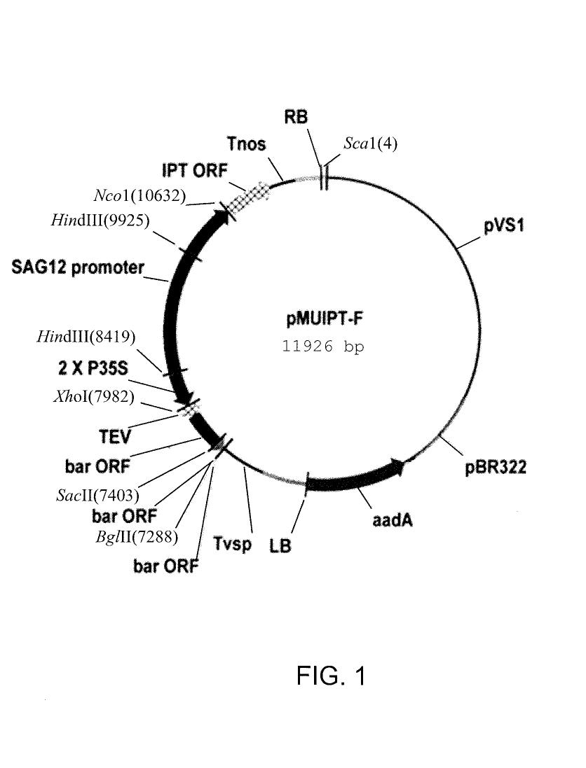

FIG. 1 is a map of binary plant transformation vector pMUIPT-F carrying the Psag₁₂-IPT expression cassette in a forward relative to the bar gene expression cassette as a representative. Either plasmid vector pMUIPT-F or pMUIPT-R contains Psag₁₂, IPT and nos: Psag₁₂ promoter, IPT ORF and nos terminator, respectively. There is also P35, TEV, bar and Tvsp: a double CaMV35S promoter, tobacco etch virus (TEV) translational enhancer, and soybean vegetative storage protein gene (VSP) terminator, respectively. LB and RB: T-DNA at the left and right borders, respectively. The remaining region has been previously described by Hajdukiewicz et al. (1994). The whole cassette of the Psag₁₂-IPT was then confirmed by sequence analysis in both the pMUIPT-F and pMUIPT-R vectors. The two vectors were then mobilized into Agrobacterium tumefaciens strain EHA101 reported by Hood et al. (1986). This was done separately as to each vector using a direct DNA transfer according to the protocol of An (1988). Additionally, a control Agrobacterium was established by placing a standard control vector pZY102 into EHA101, this in place of the pMUIPT-F and pMUIPT-R vectors.

The foregoing discussion shows that the P_{SAG12}-IPT expression cassette included an auto-regulating leaf-senescence promoter (P_{SAG12}), the bacterial isopentenyl transferase gene (IPT), and a nos terminator. This P_{SAG12}-IPT expression cassette was cloned into a binary plant transformation vector pZY101 at its multiple cloning site in two opposite orientations, i.e., forward (pMUIPT-F) and reverse (pMUIPT-R), respectively (see FIG. 1 but only pMUIPT-F is shown).

### Example 2

### Soybean Transformation

A number of soybean genotypes including “Williams 82”, “Magellan”, and “Mustang” that represent various maturity groups were purchased from Illinois Foundation Seed, Inc of Champaign, Ill. and Missouri Foundation Seed Stock, Columbia, Mo., respectively, and used for subsequent Agrobacterium-mediated transformation with the pMUIPT-F and pMUIPT-R vectors. Soybean transformation process followed the protocol described previously (Zhang et al., 1999; Zhang et al., 2000; Zeng et al., 2004); however, antioxidants DTT and sodium thiosulfate were added to the co-cultivation medium, each at 1 mM final concentration as previously reported by Olhoft et al. (2003). In addition, various levels and schemes of herbicide glufosinate selections were evaluated during shoot initiation and elongation stages. The following discussion describes those procedures in greater detail:

Seed germination: Soybean seeds were surface-sterilized by an overnight exposure to chlorine gas as practiced by Di et al. (1996). Sterilized seeds were germinated in 100×20 mm Petri plates containing Gamborg's B5 basal medium at a pH 5.8, as practiced by Gamborg et al. (1968). The medium was supplemented with 2% sucrose. The plates were stacked 5 high and placed in plastic bags which were cut to provide four slits each two inches long. Seeds were germinated for 5 days in a growth room at 24° C. using 18/6 photoperiod, and with light intensity at 150-200 μE/s. The light source was cool white florescent light (Sylvania or Industrial model #F48T12/CW/VHO).

Agrobacterium manipulation: Agrobacterium EHA101 cultures were grown in YEP medium (10 g/L peptone, 5 g/L yeast extract, and 5 g/L NaCl, pH7.0) supplemented with proper antibiotics (25 mg/L chloramphenicol, 50 mg/L kanamycin, spectinomycin 100 mg/L, and streptomycin 100 mg/L) to an OD₆₅₀=1.1 to 1.2 at 28° C. with shaking at 250 rpm. The bacterial cultures were then centrifuged at 3500 rpm for 10 min and pallets were suspended to a final OD₆₅₀=0.4-1.2 in inoculation/resuspension medium. The inoculation/resuspension medium contained 1/10 Gamborg's B5 salts (pH5.4), and full strength of B5 vitamin, 3% sucrose, 20 mM MES, 1.7 mg/L BAP, 0.25 mg/L GA₃, 200 μM acetosyringone (AS). All growth regulators, vitamins and AS were filter-sterilized, before being added to the remaining components of the medium which was autoclaved for 20 minutes.

Inoculation: Cotyledonary explants were prepared from 5-day-old soybean seedlings by making a horizontal slice through the hypocotyl region, about 3 mm below the cotyledon. Then vertical slice was made between the cotyledons and embryonic axis was removed, generating 2 cotyledonary node explants. Each explant was then wounded using a #15 razor blade by making 5-10 slices, paralleled to hypocotyl and on the nodal region of the explant. Each slice was about 3 mm long and 0.5 mm in depth covering the cotyledon/hypocotyl junction. Before wounding was made, the blade was dipped into the Agrobacterium inoculums. Explants were then immersed in the Agrobacterium inoculums for 30 min and then transferred onto 100×15 Petri dish containing co-cultivation medium. This medium contained Agrobacterium inoculation/resuspension medium amended with 3.3 mM L-cysteine, 1 mM DTT, and 1 mM sodium thiosulfate, solidified with 0.5% washed agar, and overlaid with a piece of sterile Whatman #1 filter paper as practiced by Mullins et al. (1990) and Zhang et al. (1997). The explants (5 per plate) were cultured, flat face down, on this medium for 5 days at 24° C. under 18/6 photoperiod with a light intensity of 150-200 μE/s. The co-cultivation plates were wrapped with parafilm.

Shoot initiation: After co-cultivation, explants were briefly washed in B5 washing medium (pH5.7). The medium is composed of Gamborg's B5 salts and vitamins, 1.7 mg/L BAP, 3% sucrose, 3 mM MES, 100 mg/L cefotaxime, 50 mg/L timentin, 50 mg/L vancomycin. All antibiotics and vitamins were filter-sterilized before added to the autoclaved remaining medium. Following wash, explants were transferred to shoot initiation (SI) plates (100×20 mm) containing the B5 medium which were identical to the washing medium except that medium was solidified with 0.3% Phytagel™ (Sigma-Aldrich, USA) and with 0-5 mg/L glufosinate. The plates were wrapped with 3M venting tape (Scotch™, 3M, USA) and placed under the culture conditions as outlined above for co-cultivation. After 2 weeks of culture on SI medium, hypocotyl regions of the explants were removed and remaining tissues with differentiating node were subcultured onto fresh SI medium amended with 0-10 mg/L glufosinate (AgrEvo, USA).

Shoot elongation and rooting: Following an additional 2 weeks of culture, the cotyledons of the explants were removed and remaining tissues with differentiating shoots and buds were transferred to shoot elongation (SE) medium (pH5.7). The SE medium was composed of MS basal salts as practiced by Murashige and Skoog (1962), B5 vitamins, 3% sucrose, 3 mM MES, 1 mg/L trans-zeatin riboside, 0.5 mg/L GA₃, 0.1 mg/L IAA, 50 mg/L glutamine, 50 mg/L asparagin, 100 mg/L cefotaxime, 50 mg/L timentin, 50 mg/L vancomycin, 3-5 mg/L glufosinate,and 0.3% Phytagel™ (Sigma, Chemical, USA) The explants were subcultured biweekly to freshen the SE medium until shoots reached more than 3 cm long. The elongated shoots were excised and their basal regions were soaked in 1 mg/ml IBA for 1 to 4 min before they were rooted in a 100×25 glass culture tube containing rooting medium. Root medium contains MS slats, B5 vitamins, 2% sucrose, 3 mM MES, 50 mg/L glutamine, 50 mg/L asparagine, 100 mg/L cefotaxime, 50 mg/L timentin, 50 mg/L vancomycin, 0.3% Phytagel™ and pH5.7.

Acclimatization and greenhouse care: Rooted shoots were washed briefly with distilled H₂O and transferred to jiffy pots containing Metro-mix 200 soil from Hummert International of Earth City, Mo. The growth conditions were the same as those for the SI, SE, and rooting. Hardened plantlets were then transferred to three-gallon pots containing Pre-mix soil and Osmocot fertilizer 14-14-14 (Hummert International, Earth City, Mo.) and watered as needed under greenhouse conditions.

Control: The above procedures were repeated using Agrobacterium-mediated transformation with a standard control vector pZY102 in place of the pMUIPT-F and pMUIPT-R vectors.

### Example 3

### Leaf-Painting Assay

All plant lines recovered from the transformation experiments of Example 2 were first assayed using leaf-painting as reported by Zhang et al. (1999). The assay was conducted twice at the acclimatization stage and once at greenhouse stage. For the leaf-painting assay, a 100-200 mg/L solution of the glyphosate herbicide Liberty® from Aventis CropScience (Research Triangle Park, N.C., USA) was applied onto the middle vain region of each young fully-expended leaf with a cotton swab. The results were observed 5 days later: susceptible plants showed leaf yellowing or necroses, and resistant plants showed no symptoms (FIG. 3).

Depending on genotypes, almost all IPT-transgenic Williams 82 lines were susceptible to the Liberty® herbicide. This result was in sharp contrast with the phenotypes of the IPT-transgenic Magellan and Mustang soybean, which predominantly showed Liberty® resistance. Similarly, the pZY102-transgenic lines of the control vector showed both herbicide resistance and GUS gene expression.

### Enhanced Transformation Frequency by Psag₁₂-IPT

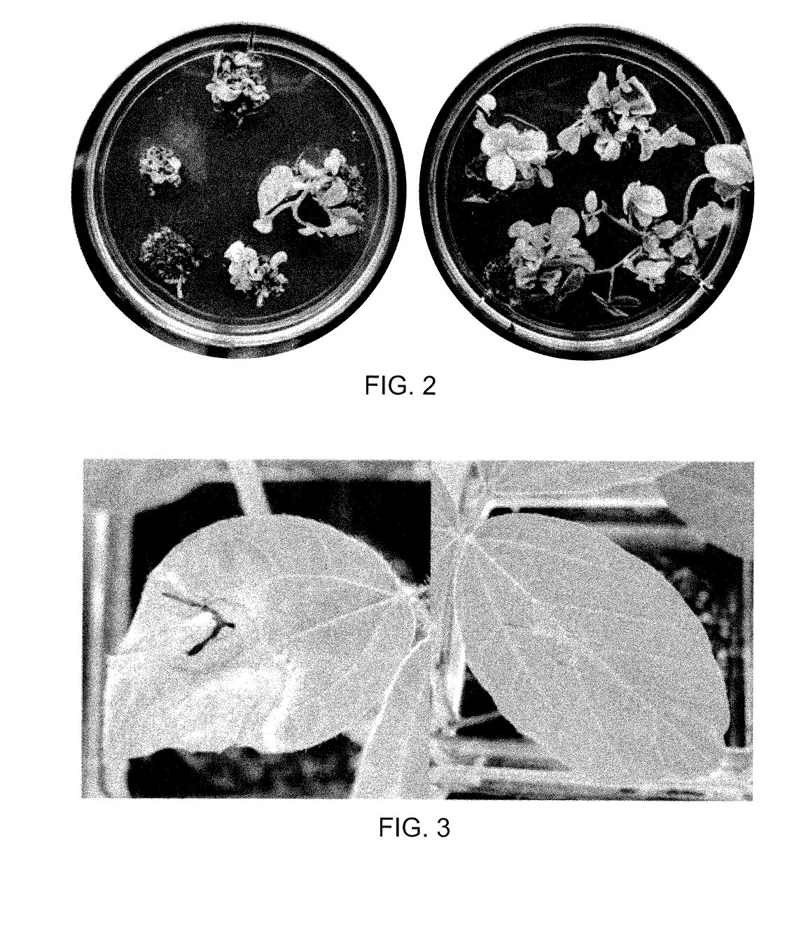

The effect of the pMUIPT-F and pMUIPT-R vector was evaluated in comparison with transformation results from the standard control vector pZY102 to assess relative transformation efficiency. These experiments screened nine soybean genotypes representing various maturity groups. FIG. 2 showed the comparative results of in vitro culture responses of a high-frequency transgenic recovery of soybean using the Psag₁₂-IPT gene expression cassette for enhancing transformation. FIG. 2 left shows the in vitro culture response of treatment using standard transformation control vector pZY102 carrying the bar gene (conferring resistance to herbicide glufosinate) and the GUS reporter gene cassette reported by Zeng et al. (2004). FIG. 2 right shows in vitro culture response of the treatment using the same vector as pZY102, except that the GUS reporter gene cassette is replaced with the Psag₁₂-IPT gene expression cassette, showing that almost every explant produces shoots which resist the herbicide glufosinate in culture medium. The use of pMUIPT-F significantly enhanced transformation (p<0.0005) in 5 out of 9 soybean genotypes screened (Table 1). The average transformation frequencies were 7.4% across the 9 genotypes and 10% across the 5 genotypes (whose transformation was improved by Psage₁₂-IPT), respectively (p<0.0001). Of these, Magellan and Mustang showed as high as 11.7% and 14% transformation frequency, respectively, representing a 2.9- and 3.8-fold increase over the standard control vector in experiments with relatively large sample size. These efficient transgenic recoveries were achieved under the selection conditions 0-10 mg/L glufosinate during the first and second shoot initiation (SI) stages and 3-5 mg/L glufosinate during shoot elongation (SE) stage. These recovery efficiencies were in sharp contrast with pZY102 standard control which showed an average of 3.2% frequency cross the 9 different genotypes. One exception is that pZY102 provides a better transformation than Psage₁₂-IPT in genotype Pana.

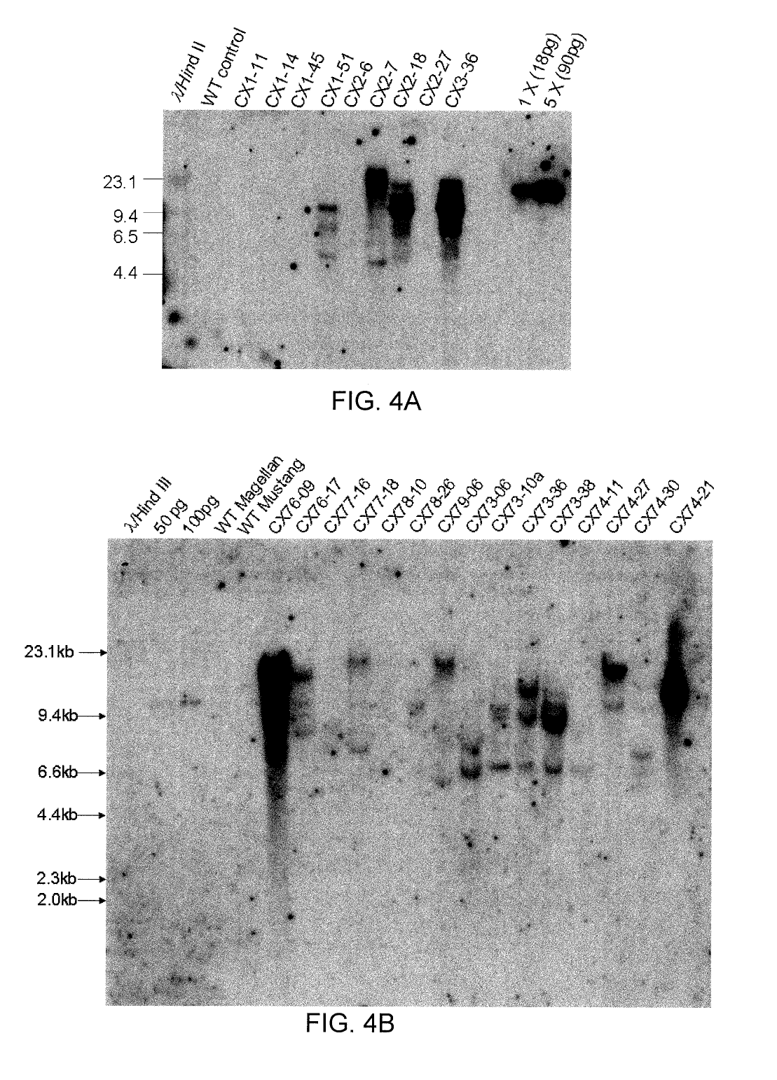

Data in Table 1 are from at least 2 independent experiments (replicates) per treatment. Each treatment deployed at least 40 explants per genotype. The experimental design was randomized complete block design (RCBD) and two different constructs were assigned to the same block. Results were subjected to ANOVA analysis using SAS PROC GLM and means were separated using Duncan's Multiple Range Test (DMRT) at α=0.01 level. Numbers (average transformation frequencies) followed by the different letters indicate a significant difference at α=0.01 level. A pair-wise comparisons in each of the 5 genotypes, i.e., Williams 82, Thorne, Linford, Magellan, and Mustang also showed significantly higher transformation frequencies using pMUIPT-F than pZY102 (Table 1 but statistics of mean comparisons not shown). The efficiency is defined as the number of independent transgenic soybean events divided by total number of explants to start with including contaminated explants. Independent events were recognized by transgenic shoot recovery from different explants and their functional expression of transgene as indicated by bar through herbicide leaf-painting (FIG. 3) followed by Southern blot confirmation of different banding patterns. Transgene insertion and other events were later verified by Southern blot analysis (FIG. 4) using random samples from a large number of recovered plants. Note that the average transformation frequency of these genotypes could have been even higher, if the seed conditions for the same experiments had been normal and experimental errors had been prevented. This is clearly indicated by a lower transformation frequency of standard control pZY102 that provide average of 5.5% efficiency in these genotypes under normal experimental conditions including normal seeds.

TABLE 1
Enhanced Agrobacterium-mediated transformation in soybean by SAG₁₂-IPT

| | | Number | | | | Liberty ® | | |
|---|---|---|---|---|---|---|---|---|
| | | of | Total | Plantlets | Plants in | resistant | Transformation | Fold |
| Vector | Genotypes | replicates | explants | in soil | greenhouse | plants | frequency (%) | increase |
| pZY102 | Williams 82 | 4 | 199 | 8 | 5 | 1 | 0.5 | |
| | Maverick | 8 | 402 | 30 | 21 | 14 | 3.5 | |
| | Pana | 2 | 80 | 5 | 3 | 2 | 2.5 | |
| | Thorne | 2 | 79 | 6 | 4 | 0 | 0.0 | |
| | Bert | 2 | 80 | 5 | 3 | 1 | 1.3 | |
| | Linford | 2 | 80 | 5 | 5 | 0 | 0.0 | |
| | Mustang | 5 | 380 | 40 | 31 | 15 | 3.9 | |
| | Magellan | 6 | 472 | 37 | 26 | 19 | 4.0 | |
| | Mini Max | 6 | 306 | 40 | 38 | 14 | 4.6 | |
| | Subtotal | | 2078 | 176 | 136 | 66 | 3.2a | |
| pMUIPT-F | Williams 82 | 4 | 183 | 20 | 20 | 3 | 1.6 | 3.3 |
| | Maverick | 8 | 398 | 90 | 72 | 16 | 4.0 | 1.2 |
| | Pana | 2 | 80 | 8 | 6 | 0 | 0.0 | — |
| | Thorne | 2 | 80 | 35 | 29 | 5 | 6.3 | >6.3 |
| | Bert | 2 | 80 | 10 | 7 | 1 | 1.3 | 1.0 |
| | Linford | 2 | 80 | 3 | 3 | 2 | 2.5 | >2.5 |
| | Mustang | 5 | 379 | 119 | 87 | 52 | 14.0 | 3.6 |
| | Magellan | 6 | 471 | 218 | 146 | 55 | 11.7 | 2.9 |
| | Mini Max | 6 | 300 | 79 | 51 | 13 | 4.3 | 0.9 |
| Total | Subtotal | | 2051 | 582 | 421 | 152 | 7.4b | 2.3 |

### Example 4

### Southern Blot Analysis

Southern blot analysis was performed according to the CTAB (Hexadecyltrimethylammonium bromide) protocol that has been previously reported by Doyle and Doyle (1990), and Poresbski et al. (1997). The protocol was modified to extract genomic DNA from young leaves of the transgenic soybean lines. Briefly, about 1 g of each young leaf sample was grinded in liquid nitrogen and samples were lysed at 65° C. for 45 min in a DNA extraction solution. The solution contained 5 parts of DNA extraction buffer (350 mM sorbitol, 100 mM Tris-HCl, 5 mM EDTA and pH8.2), 5 parts of lysis buffer (200 mM Tris base, 50 mM EDTA, 2M NaCl, and 2% (w/v) CTAB), and 1 part of 20% (w/v) sarkosyl. Samples were centrifuged and supernatants were transferred to and mixed well with 5 ml of protein precipitation solution (10M NH₄OAc). After samples were centrifuged, supernatants were mixed, at 2:1 ratio, with isopropanol. The genomic DNA was then collected with a glass hook, soaked and washed in 70% ethanol, re-suspended in TE buffer (10 mM Tris and 1 mM EDTA, pH8.0), and incubated with RNAse A for 30 min. Each sample was then extracted with a mixture of phenol:chloroform:Isoamyl alcohol (25:24:1 ratio and pH8.0) and DNA was precipitated in the presence of 50% isopropanol and 0.1M NaOAc. The DNA pallet was washed with 70% ethanol, air-dry, and re-suspended in de-ionized and distilled (dd) H₂O before use.

Twenty to thirty μg of genomic DNA from each plant sample was digested with restriction enzyme, electroporesed on 1% (w/v) agarose gel, and blotted onto the Zeta-Probe-GT membrane (BioRad, Richmond, Calif.) in 20× sodium citrate and sodium chloride (SSC) transfer buffer overnight by capillary transfer. The membranes were hybridized for 16-18 hours with various DNA probes. Probes were obtained by a gel purification fragment containing the probe of interest, then labeling it with ³²P-dCTP using a random-prime-labeling kit (Stratagene, La Jolla, Calif.). Pre-hybridization and hybridization solutions contained 6×SSC, 40 mM M NaPO₄ (pH7.2), 1% (w/v) sodium dodecyl sulfate (SDS), 0.1 mg/ml Herring sperm DNA, and 3.25× Denhart solution. After hybridization, membranes were rinsed once with rinse solution (2×SSC, 0.4% SDS) followed by two washes (1×SSC and 0.25% SDS; 0.5×SSC and 0.25% SDS), respectively. The temperatures for pre-hybridization, hybridization, and washes were 60° C. or 65° C., according to the probes used. For repeated probing of membranes, the incorporated ³²P-dCTP was removed following the protocols as described in Sambrook et al. (1989). The Sambrook procedure was modified by washing the membrane in a first wash solution (0.1N NaOH, 0.2% SDS) and a second wash solution (0.2M Tris (pH7.5), 0.1×SSC, 0.2% SDS) at room temperature for 20 min each.

On average, approximately 50% of recovered Williams 82, Mustang and Magellan independent lines were positive on Southern blot analysis and showed different bandings as detected by IPT probe, suggesting stable integration and independent events. The per-genome copy number of the transgene inserts was estimated from their band intensity by comparison with 1× and 5× genomic equivalent copy control of vector pMUIPT-F. This is shown, for example, in FIG. 4, which show the results of genomic Southern blot analysis. FIG. 4 resulted from a Bgl II digest of genomic DNA from “Williams 82” (A) as well as Magellan and Mustang (B) transgenic lines and indicated the copy number of IPT transgene inserts per genome locus. The scale on the left indicates size of DNA ladder in kb. The lane descriptions are as follows: FIG. 4A: Lane 1, λ/Hind III DNA ladder; Lane 2, wild-type control; Lanes CX1-11 to CX3-36 are recovered soybean among which about four turn out to be transgenic; Lanes 13 and 14, 1× and 5× genome equivalent representing 1 copy and 2 copy number controls, respectively, using 18 and 50 pg of pMUIPT-F plasmid DNA. FIG. 4B: Lane 1, λ/Hind III DNA ladder; Lanes 3 and 4, blot sensitivity controls using 50 and 100 pg of pMUIPT-F plasmid DNA; Lane 4 and 5, wild type Magellan and Mustang controls; Lanes CX76-09 to CX79-06, transgenic Magellan events; Lanes CX73-06 to CX74-21, transgenic Mustang events. The membrane was probed with Psag₁₂-IPT cassette.

It is possible that the native soybean genome might contain endogenous IPT gene homologs, although the IPT gene has not been found in most plant species. Nine IPT gene homologs have been recently identified from Arabidopsis, as reported by Kakimoto (2001), Takei et al. (2001) and Sun et al. (2003). Our Southern blot results here didn't show endogenous IPT sequence when the bacterial IPT open-reading frame was used as a probe under a stringent hybridization condition of 65° C. Therefore, it is likely that endogenous IPT share low degree of sequence identity with bacterial IPT gene counterpart. Bgl II was used to digest T₁ genomic DNA. This was done to distinguish different transgene loci by different bandings since this restriction enzyme cut only once within the T-DNA region of the vector (see FIG. 1).

The discrepancy between herbicide resistance and Southern blot analysis in other soybean genotypes has been attributed to the possibility that the bar gene may have been silenced. Further analysis of transgenic lines showing herbicide susceptible should shed some lights on this hypothesis.

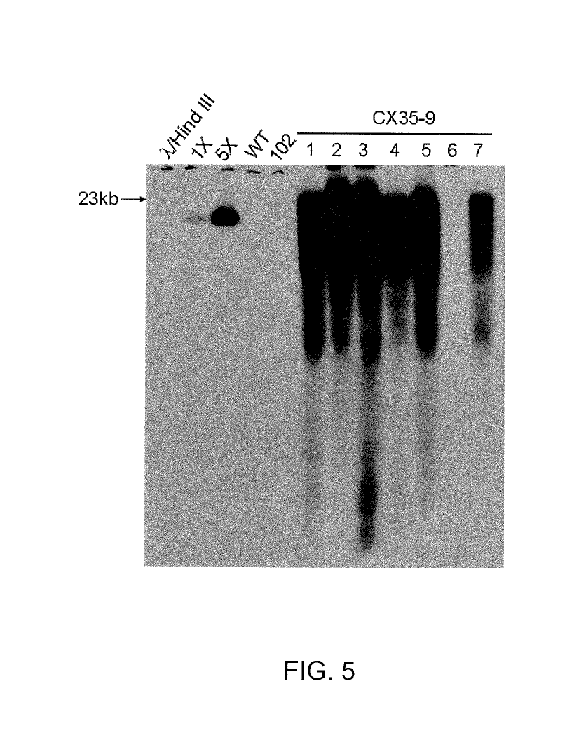

Progeny analysis was further conducted to confirm stable inheritance of transgenes. FIG. 5 illustrates such Southern blot assay using a random set of progeny from transgenic Magellan event CX35-9 with IPT ORF as hybridization probe (FIG. 5). The result clearly showed that IPT transgene had transmitted to the progeny successfully. Note: lambda/Hind III, DNA ladder; 1× and 5×, 1× and 5× genome equivalent copy number controls, respectively, using pMUIPT-F plasmid DNA; WT, wild type soybean control; 102, transgenic pZY102 soybean control; 1 to 7 are the progeny plants of event CX35-9.

The results shown above in the Examples, with an average of 11.7% to 14% using the bar gene as negative selectable marker, represent the highest transgenic recovery in any soybean transformation efforts that have been reported to date. In comparison, the average transformation frequency of the latest Agrobacterium-mediated soybean cotyledonary node system was 3-5% for a number of soybean genotypes, when bar gene was used as a selectable marker as reported by Zeng et al. (2004) and Paz et al. (2004). It will be appreciated that the highest transformation efficiency (16%) that is reported in the prior art discussed above involves use of the soybean genotype “Bert”, a maturity group “0” (grown only in high latitude) employing HPT II as a selectable marker (Olhoft et al. 2003).

These results demonstrate that expression from the IPT gene under autoregulated conditions has a profound impact upon T-DNA transformation. Without being bound by theory, one possible explanation for this effect is that expression of the IPT gene under autoregulated condition may provide optimal levels of cytokinins for transgenic differentiating tissues, and this may enhance the frequency of successful transformation. The plant endogenous IPT gene-encoded protein, IPT, regulates a first committed step in plant cytokinin pathway that is responsible for the biosynthesis of all types of cytokinin. This IPT-regulated pathway may have a less crucial role in wild type soybean for morphogenesis, but may play an essential role in adventitious shoot induction and subsequent plant morphogenesis in transformed explant tissues.

The high-frequency transformation reported here suggests that auto-regulated expression of the IPT gene plays an important role in soybean transformation. Agrobacterium tumefaciens has co-evolved with its host plant and carries within its T-DNA the IPT gene. These results further suggest that under vir gene induction conditions Agrobacterium may have a surprising capability of delivering its T-DNA, as enhanced by this Agrobacterium IPT gene. At a minimum, these results show that expression of the IPT gene influences the cell cycle progression of the host plant.

One earlier study on monitoring genome-wide gene expression using IPT-transformed plants showed that expression of bacterial IPT gene up- and down-regulated over 823 and 917 genes, respectively, in transgenic plants, as reported by Hoth et al. (2003). The study found that none of these genes, whose levels are influenced by IPT, control plant chromatin remodeling. Interestingly, one of these genes that was up-regulated, CycD3, is a plant cell cycle gene encoding cyclin3 protein whose function is to shift the cell phase from G1 to S, a critical transition stage when nuclear chromosomes are most accessible to T-DNA integration. Therefore, one conclusion that may be drawn from the foregoing results is a possible role of the IPT gene in activation of cell division through the D-type cyclin, for example, the D-type cyclin as discussed Soni et al. (1995); Dewitte et al. (1996); and Riou-Khamlichi et al. (1999). The result of this activation is the enhanced accessibility of plant chromosomal regions to the T-DNA integration. This conclusion is further supported by the finding that RepA, a germini-virus gene that controls plant cycle, was capable of substantiating maize transformation by changing the cell phase from G1 to S, as reported by Gordon-Kamm et al. (2002).

Table 2 lists additional genes affecting cell cycle and autoregulating promoters from known genes affecting cytokinin, auxin, and sugar pathways in plant. Autoregulating promoters are those promoters that are regulated through a feedback loop such that the level of their activation is tied to the level of the protein whose expression is under control of the promoter. Thus, the expression level of a protein under control of an autoregulating promoter may be more fine-tuned than the level of those under control of a constitutive promoter or an inducible promoter. Additional auto-regulating promoters may also be found in those affecting plant developmental pathway genes or even from other organisms. Although these genes and promoters have not been reported to be useful for transformation enhancement, these autoregulating promoters may be used, either directly or after modification, to drive the IPT gene, other cytokinin synthesis related genes, or other plant developmental pathway genes for the enhancement of the plant transformation. The promoters shown in Table 2 are autoregulating promoters, and the genes are those affecting plant pathways. These promoters may be coupled with the ORFs from the genes to control expression of the ORFs using standard techniques of recombinant DNA technology, for example, as are known from a perspective of ordinary skill and exemplified in the Examples and the discussion above.

In yet another aspect, the IPT gene may be placed under the control of either a constitutive or a tissue specific promoter to enhance regeneration and transformation of soybean. Although there has been no report that IPT under of the control of tissue specific promoter enhances plant regeneration and transformation, the use of constitutive promoter driving IPT expression has been shown to promote tobacco and sunflower transformation, respectively (Endo et al., 2002; Molinier et al., 2002).

TABLE 2
Cell cycle genes and auto-regulating promoters potentially useful for
enhancement of plant transformation

| Sources of origin | Candidate genes or promoters | References |
|---|---|---|
| Plant cell cycle genes | CycD3 | Jacqmard et al. (1994) |
| Plant receptor/signal | CBPs, CKI1, ARR, and GCR1 | Mok and Mok (2001) for |
| transduction genes | | review |
| Plant promoter | MAP kinase and cyclin B gene | Sheriff et al. (2003) |
| | promoters | |
| | CrRR1 promoter | Papon et al. (2003) |
| | maize opaque-2 promoter | Rossi et al. (1997) |
| | rice phytochrome gene promoter | Kay et al. (1989) |
| | Arabidopsis ABI3, ABI4, and ABI5 | Brocard et al. (2002) |
| | gene promoters | |
| Bacterial promoter | luxC gene promoter | Miyamoto et al. (1990) |
| | bvgₚ₁ and bvgₚ₂ promoters | Roy et al. (1990) |
| | pasA, pasB, pasC, and parD gene | Smith and Rawlings et |
| | promoters | al. (1998) |
| Mammalian promoter | TK promoter | Strathdee et al. (1999) |
| Viral promoter | Cp gene promoter | Schlager et al. (1996) |

It is surprising that very little functional expression from the bar gene was detected among most of the IPT-transgenic Williams 82 lines and many other soybean genotypes, as shown by the leaf-painting assay. The pMUIPT-F lines displayed herbicide susceptible phenotypes, so this rules out the possibility that an aberrant RNA was generated due to the transcriptional read-through from the Psag₁₂-IPT cassette.

As confirmed by these results, it is likely that expression of the IPT gene influences the expression status of the bar gene, or vice versa. This is because that gene order and orientation of adjacent genes within the same T-DNA may influence the expression of each other, as observed by Unger et al. (2001). Unger et al. reported that the expression of herbicide resistance PAT gene was reduced but expression of its upstream gene (male-sterile gene) was enhanced, causing increased phenotypes (male sterility). This difference in gene expression was further increased when the male sterile gene cassette was placed upstream of the PA T gene cassette. This was the case in the experiments described in the Examples, where the IPT gene was indeed placed upstream of the bar gene cassette.

It is also conceivable that ectopic expression of the IPT gene will up- and down-regulate the expression of numerous endogenous genes in transgenic plants, complicating gene expression analysis. Therefore, it is desirable to remove the IPT transgene from its recipient plants once the transgenic plants have been recovered. This may be accomplished by constructing a plant transformation vector that carries two T-DNA regions, wherein the first T-DNA region contains the P_{SAG12}IPT cassette that will be co-integrated into T₀ plant genome with a “gene of interest” located in the second T-DNA region. The _{SAG12}IPT cassette will be subsequently removed from the plant genome of some progeny lines during T₀ meiosis stage, leading to generation of some marker-free T₁ transgenic progeny. A similar type of looping strategy has been used with success, for example, as reported in Depicker et al. (1985); Komari et al. (1996); and Xing et al. (2000).

All cited references, including patents, patent applications, scientific publications, etc, are incorporated by reference to the same extent as though fully replicated herein.

### LIST OF CITED REFERENCES

The following references are incorporated by reference to the same extent as though fully replicated herein:
Amasino R M, Gan S. Noh Y S. 1997. Transgenic plants with altered senescence characteristics. U.S. Pat. No. 6,359,197. Wisconsin Alumni Research Foundation (Madison, Wis.).
An G, Event P R, Mitra A, Ha S B. 1988. Binary vectors. In: Gelvin S B, Schilperoort R A (Eds), Plant Molecular Biology Manual A3, pp 1-19. Kluwer Academic Publishers, Dordrecht.
Aoyama T, Chua N H. 1997. A glucocorticoid-mediated transcriptional induction system in transgenic plants. Plant J 11:605-612.
Bonnard G, Tinland B, Paulus F, Szegedi E, Otten L. 1989. Nucleotide sequence, evolutionary origin and biological role of a rear-ranged cytokinin gene isolated from a wide host range biotype III Agrobacterium strain. Mol. Gen. Genet. 216:428-438.
Brocard I M, Lynch T J, Finkelstein R R. 2002. Regulation and role of the Arabidopsis Abscisic Acid-Insensitive 5 gene in abscisic acid, sugar, and stress response. Plant Physiol 129:1533-1543.
Carrington J, Freed D D. 1990. Cap-independent enhancement of translation by a plant potyvirus 5′ nontranslated region. J Virol 64:1590-1597.
Chen C. Cytokinin biosynthesis and interconversion. 1997. Physiol Plant 101:665-673.
Clemente T E, LaVallee B J, Howe A R, Conner-Ward D, Rozman R J, Hunter P E Broyles D L, Kasten D S, Hinchee M A. 2000. Progeny analysis of glyphosate selected transgenic soybeans. Crop Sci 40: 797-803
Depicker G, Herman L, Jacobs A, Schell J, Van Montagu M. 1985. Frequencies of simultaneous transformation with different T-DNAs and their relevance to Agrobacterium/plant cell interaction. Mol Gen Genet 201:477-484.
Di R, Purcell V, Collins G B, Ghabrial S A (1996) Production of transgenic soybean lines expressing the bean pod mottle virus coat protein precursor gene. Plant Cell Rep 15:746-750.
Dewitte W, Riou-Khamlichi C, Scofield S, Healy J M, Jacqmard A, Kilby N J, Murray J A. Altered cell cycle distribution, hyperplasia, and inhibited differentiation in Arabidopsis caused by the D-type cyclin CYCD3. Plant Cell. January 2003;15(1):79-92.
Doyle, Doyle. 1990. Isolation of plant DNA from fresh tissue. Focus 12:13-15.
Ebinuma H, Sugita K, Matsunaga E, Yamakado M. 1997. Selection of marker-free transgenic plants using isopentenyl transferase gene. Proc Natl Acad Sci USA 94:2117-2121.
Endo S, Kasahara T, Sugita K, Ebinuma H. 2002. A new GST-MAT vector containing both ipt and iaaM/H genes can produce marker-free transgenic tobacco plants with high frequency. Plant Cell Rep. 20: 923-928.
Enriquez-Obregon G A, Prieto-Samsonov D L, de la Riva G A, Perez M, Selman-Housein G, Vazquez-Padron R I (1999) Agrobacterium-mediated Japonica rice transformation: a procedure assisted by an antinecrotic treatment. Plant Cell Tiss Organ Cult 59:159-168.
Enriquez-Obregon G A, Vazquez-Padron R I, Prieto-Samsonov D L, de la Riva G A, Selman-Housein G (1998) Herbicide-resistant sugarcane (Saccharum officinarum L.) plants by Agrobacterium-mediated transformation. Planta 206:20-27.
Frame B, Shou H, Chikwamba R, Zhang Z, Xiang C, Fonger T, Pegg S E, Li B, Nettleton D, Pei D, Wang K (2002) Agrobacterium-mediated transformation of maize embryos using a standard binary vector system. Plant Physiol 129: 13-22.
Frame B R, McMurray J M, Fonger T M, Main M L, Taylor K W, Torney F J, Paz M M, Wang K. (2006) Improved Agrobacterium-mediated transformation of three maize inbred lines using MS salts. Plant Cell Rep 25:1024-1034.
Gamborg O L, Miller R A, Ojima K. 1968. Nutrient requirements of suspension cultures and soybean root cells. Exp Cell Res 50:151-158.
Gordon-Kamm W, Dilkes B P, Lowe K, Hoerster G, Sun X, Ross M, Church L, Bunde C, Farrell J, Hill P, Maddock S, Snyder J, Sykes L, Li Z, Woo Y M, Bidney D, Larkins B A. 2002. Stimulation of the cell cycle and maize transformation by disruption of the plant retinoblastoma pathway. Proc Natl Acad Sci USA 99:11975-11980.
Hanin M, Paszkowski J. 2003. Plant genome modification by homologous recombination. Curr Opin Plant Biol. 6(2):157-162.
Hinchee M A W, Connor-Ward D V, Newell C A, McDonnell R E, Sato S J, Gasser C S, Fischhoff D A, Re D B, Fraley R T, Horsch R B. 1988. Production of transgenic soybean plants using Agrobacterium-mediated gene transfer. Bio/Technol. 6:915-922.
Hiei Y, Ohta S, Komari T, Kumashiro T. 1994. Efficient transformation of rice (Oryza sativa L.) mediated by Agrobacterium and sequence analysis of the boundaries of the T-DNA. Plant J. 6: 271-282.
Hiei Y, Ohta S, Komari T, Kubo T. 1997. Transformation of rice mediated by Agrobacterium tumefaciens. Plant Mol. Biol. 35:205-218.
Hood E E, Helmer G L, Fraley R T, Chiton M D. 1986. The hypervirulence of Agrobacterium tumefaciens A281 is encoded in a region of pTiBo542 outside of T-DNA. J Bacteriol 168:1291-1301.
Hoth S, Ikeda Y, Morgante M, Wang X, Zuo J, Hanafey M K, Gaasterland T, Tingey S V, Chua N H. 2003. Monitoring genome-wide changes in gene expression in response to endogenous cytokinin reveals targets in Arabidopsis thaliana. FEBS Lett. 554:373-380.
Ishida Y, Saito H, Ohta S, Hiei Y, Komari T, Kumashiro T. 1996. High efficiency transformation of maize (Zea mays L.) mediated by Agrobacterium tumefaciens. Nature Biotechnol. 14:745-750.
Jacqmard A, Houssa C, Bernier G. 1994. Regulation of the cell cycle by cytokinins. In: Cytokinins-Chemistry, activity, and function. Boca Raton: CRC Press, pp. 197-215.
Jefferson R A, Kavanagh T A, Bevan M W. 1987. GUS fusions: β-glucuronidase as a sensitive and versatile gene fusion marker in higher plants. EMBO J 6:3901-3907.
Kakimoto T. 2001. Identification of plant cytokinin biosynthetic enzymes as dimethylallyl diphosphate:ATP/ADP isopentenyltransferases. Plant Cell Physiol. 42:677-685.
Kay S A, Keith B, Shinozaki K, Chye M L, Chua N H. 1989. The rice phytochrome gene: structure, autoregulated expression and binding of GT-1 to a conserved site in the 5′ upstream region. Plant Cell 1:351-360.
Komari T. 1990. Transformation of cultured cells of Chenopodium quinoa by binary vectors that carry a fragment of DNA from virulence region of pTiBo542. Plant Cell Rep. 9: 303-306.
Komari T, Hiei Y, Saito Y, Murai N, Kumashiro T. 1996. Vectors carrying two separate T-DNAs for co-transformation of higher plants mediated by Agrobacterium tumefaciens and segregation of transformants free from selection markers. Plant J 10:165-174.
Kunkel T, Niu Q W, Chan Y S, Chua N H. 1999. Inducible isopentenyl transferase as a high-efficiency marker for plant transformation. Nat Biotech 17:916-919.
Li S, Li T, Kim W D, Kitaoka M, Yoshida S, Nakajima M, Kobayashi H., Characterization of raffinose synthase from rice (Oryza sativa L. var. Nipponbare). Biotechnol Lett. April 2007;29(4):635-40.
Lohman K N, Gan S, John M C, Amasino R M. 1994. Molecular analysis of natural leaf senescence in Arabidopsis thialiana. Physiol Plant 92:322-328.
Mason H S, DeWald D, Mullet J E. 1993. Identification of a methyl jasmonate-responsive domain in the soybean vspB promoter. Plant Cell 5:241-251.
McCabe M S, Garratt L C, Schepers F, Jordi W J R M, Stoopen G M, Davelaar E, Rhijn H A V, Power J B, Davey M R. 2001. Effects of P_{SAG12}-IPT gene expression on development and senescence in transgenic lettuce. Plant Physiol 127:505-516.
McGaw, B A, Burch L R. 1995. Cytokinin biosynthesis and metabolism. In: Davies P J, ed. Plant Hormones: Physiology, Biochemistry and Molecular Biology. 2^{nd} Ed. Dordrecht, The Netherlands, Kluwer Academic Publishers; p. 98-117.
Miyamoto C M, Meighen E A, Graham A F. 1990. Transcriptional regulation of lux genes transferred into Vibrio harveyi. J Bacteriol 172:2046-2054.
Mok D W S, Mok M C. 2001. Cytokinin metabolism and action. Annu Rev Plant Physiol Plant Mol Biol 52:89-118.
Molinier J, Thomas C, Brignou M, Hahne G. 2004. Transient expression of ipt gene enhances regeneration and transformation rates of sunflower shoot apices (Helianthus annuus L.). Plant Cell Rep. 21: 251-256.
Mullins M G, Archie-Tang F C, Facciotti D. 1990. Agrobacterium-mediated genetic transformation of grapevines: transgenic plants of Vitis rupestris scheele and buds of Vitis vinifera L. Bio/Technol 8:1041-1045.
Murashige T, Skoog F. 1962. A revised medium for rapid growth and bioassays with tobacco tissue cultures. Physiol Plant 15:473-479.
Nagy A. 2000. Cre recombinase: the universal reagent for genome tailoring. Genesis. 26(2):99-109.
Napier, J A, The production of unusual fatty acids in transgenic plants. Annu Rev Plant Biol. 2007;58:295-319.
Odell J T, Nagy F, Chua N H. 1985. Identification of DNA sequences required for activity of the cauliflower mosaic virus 35S promoter. Nature 6:810-812.
Olhoft P M, Flagel L E, Donovan C M. Somers D. 2003. Efficient soybean transformation using hygromycin B selection in the cotyledonary-node method. Planta 216:723-735.
Olhoft P M, Lin K, Galbraith J, Nielsen N C, Somers D A 2001a. The role of thiol compounds increasing Agrobacterium-mediated transformation of soybean cotyledonary-node cells. Plant Cell Rep 20:731-737.
Olhoft P M, Somers D A. 2001b. L-Cysteine increases Agrobacterium-mediated T-DNA delivery into soybean cotyledonary-node cells. Plant Cell Rep 20:706-711.
Ow D W. 2002. Recombinase-directed plant transformation for the post-genomic era. Plant Mol Biol. 48(1-2):183-200.
Papon N, Clastre M, Gantet P, Rideau M, Chénieux J C, Créche J. 2003. Inhibition of the plant cytokinin transduction pathway by bacterial histidine kinases inhibitors in Catharanthus roseus cell cultures. FEBS Lett. 537:101-105.
Paz M, Shou H, Guo Z, Zhang Z, Banerjee A, Wang K. 2004. Assessment of conditions affecting Agrobacterium-mediated soybean transformation using the cotyledonary node explant. Euphytica 136:167-179.
Pereira A. 2000. A transgenic perspective on plant functional genomics. Transgenic Res. 9(4-5):245-260.
Perl A, Lotan O, Abu-Abied M, Holland D. 1996. Establishment of an Agrobacterium-mediated transformation system for grape (Vitis vinifera L.): the role of antioxidants during grape-Agrobacterium interactions. Nat Biotechnol 14:624-628.
Poresbski S L, Bailey G, Baum R B. 1997. Modification of CTAB DNA extraction protocol for plant containing high molecular and poly-phenol components. Plant Mol Biol Reptr 12:8-15.
Ross M C, Church L A, Hill P M, Gordon-Kamm W J, Lowe K S, Hoerster G J, Bidney D L. 2003. Methods for enhancing plant transformation frequencies. U.S. patent. application Ser. No. 613,094. Pioneer Hi-Bred International, Inc. (Des Moines, Iowa).
Rossi V, Motto M, Pellegrini L. 1997. Analysis of the methylation pattern of the maize opaque-2 (O2) promoter and in vitro binding studies indicate that the O2 B-Zip protein and other endosperm factors can bind to methylated target sequences. J Biol Chem 272:13758-13765.
Roy C R, Miller J F, Falkow S. 1990. Autoregulation of the Bordetella pertussis bvgABC operon. Proc Natl Acad Sci USA 87:3763-3767.
Riou-Khamlichi C, Huntley R, Jacqmard A, Murray J A. Cytokinin activation of Arabidopsis cell division through a D-type cyclin. Science. 1999, 283(5407):1541-4.
Sambrook J, Fritsch E F, and Maniatis T. 1989. Molecular Cloning: A Laboratory Manual. 2^{nd} ed. Cold Spring Harbor Laboratory, Cold Spring Harbor, N.Y.
Schlager S, Speck S H, Woisetschlager M. 1996. Transcription of the Epstein-Barr virus nuclear antigen 1 (EBNA1) gene occurs before induction of the BCR2 (Cp) EBNA gene promoter during the initial stages of infection in B cells. J Virol 70:3561-3570.
Sheriff S T, Horswill M A, Chen C M. 2003. Effect of cytokinin on the expression of MAP kinase, cdc2, and cyclin B genes in cytokinin-heterotrophic and -autotrophic strains of Nicotiana tabacum tissues. Annu Meet Amer Soc Plant Biol. Honolulu, Hi.
Smith A S G, Rawlings D E. 1998. Autoregulation of the pTF-FC2 protein poison-antidote plasmid addiction system (pas) is essential for plasmid stabilization. J Bacteriol 180:5463-5465.
Somers D A, Makarevitch I. 2004. Transgene integration in plants: poking or patching holes in promiscuous genomes. Curr Opin Biotechnol. 15:126-131
Soni R, Carmichael J P, Shah Z H, Murray J A. A family of cyclin D homologs from plants differentially controlled by growth regulators and containing the conserved retinoblastoma protein interaction motif. Plant Cell. 1995 7(1):85-103.
Stacey G, Vodkin L, Parrott W A, Shoemaker R C. 2004. National science foundation-sponsored workshop report. Draft plan for soybean genomics. Plant Physiol 135:59-70.
Strabala T J, Bednarek S Y, Bertoni G, Amasino R M. 1989. Isolation and characterization of an ipt gene from the Ti plasmid Bo542. Mol Gen Genet 216:388-394.
Strathdee C A, McLeod M R, Hall J R. Efficient control of tetracycline-responsive gene expression from an autoregulated bi-directional expression vector. Gene 229:21-29.
Sun J, Niu Q W, Tarkowski P, Zheng B, Tarkowska D, Sandberg G, Chua N H and Zuo J. 2003. The Arabidopsis AtIPT8/PGA22 gene encodes an isopentenyl transferase that is involved in de novo cytokinin biosynthesis. Plant Physiol. 131:167-176.
Takei K, Sakakibara H and Sugiyama T. 2001. Identification of Genes Encoding Adenylate Isopentenyltransferase, a Cytokinin Biosynthesis Enzyme, in 7.4 Arabidopsis thaliana. J. Biol. Chem. 276:26405-26410.
Thompson C J, Movva N R, Tichard R, Crameri R, Davies J E, Lauwereys M. 1987. Characterization of the herbicide-resistance gene bar from Streptomyces hygroscopicus. EMBO J 6:2519-2523.
Unger E, Betz S, Xu R J, Mark Cigan A. 2001. Selection and orientation of adjacent genes influences DAM-mediated male sterility in transformed maize. Transgenic Res 10:409-422.
Vega J, Yu W, Kennon A, Chen X, Zhang Z. 2007. Improvement of Agrobacterium-mediated transformation in Hi-II maize (Zea mays L.) using standard binary vectors. Plant Cell Reports (online first: DOI 10.1007/s00299-007-0463-z).
Xing A, Zhang Z, Sato S, Staswick P, Clemente T. 2000. The use of the two T-DNA binary system to derive marker-free transgenic soybeans. In Vitro Cell Dev Biol-Plant 36:456-463.
Zeng P, Vadnais D, Zhang Z, Polacco J. 2004. Refined glufosinate selection in Agrobacterium-mediated transformation of soybean [Glycine max (L.) Merr.]. Plant Cell Rep 22:478-482.
Zhang Z, Coyne D P, Mitra A. 1997. Factors affecting Agrobacterium-mediated transformation of common bean. J. Am. Soc. Hort. Sci. 122:300-305.
Zhang Z, Coyne D P, Vidaver A K, Mitra A. 1998. Expression of human lactoferrin cDNA confers resistance to Ralstonia solanacearum in transgenic tobacco plants. Phytopathol. 88:730-734.
Zhang Z, Guo Z, Shou H, Pegg S E, Clemente T E, Staswick P E, Wang K. 2000. Assessment of conditions affecting Agrobacterium-mediated soybean transformation and routine recovery of transgenic soybean. In: Plant Genetic Engineering: Toward the Third Millennium, A. D. Arenciba (Ed), Elsevier Science B. V. (Amsterdam), pp 88-94.
Zhang Z, Xing A, Staswick P, Clemente T E. 1999. The use of glufosinate as a selective agent in Agrobacterium-mediated transformation of soybean. Plant Cell Tissue Organ Cult. 56:37-46.
Zhao Z Y, Cai T, Tagliani L, Miller M, Wang N, Pang H, Rudert M, Schroeder S, Hondred D, Seltzer J, Pierce D. 2000. Agrobacterium-mediated sorghum transformation. Plant Mol Biol. 44:789-798.
Zhao Z Y, Gu W, Cai T, Pierce D A. 1999. Methods for Agrobacterium-mediated transformation. U.S. Pat. No. 5,981,840.
Zhao Z Y, Gu W, Cai T, Pierce D A. 2004. Methods for Agrobacterium-mediated transformation. U.S. Pat. No. 6,822,144.
U.S. patent application Ser. No. 11/835,328 entitled “LysM Receptor-Like Kinases To Improve Plant Defense Response Against Fungal Pathogens.”

1. A method of transforming a plant with a transgene of interest, the method comprising the steps of:
(a) constructing a vector comprising:
an autoregulating promoter coupled with an open reading frame (ORF) to control the expression thereof,
the open reading frame coding for the expression of a polypeptide affecting at least one plant cell cycle pathway selected from the group consisting of cytokinin, auxin, and sugar pathways, and
a transgene of interest;
(b) incorporating the vector into an Agrobacterium; and
(c) using the Agrobacterium to transform a plant.

2. The method of claim 1, wherein the plant is soybean.

3. The method of claim 1, further comprising a step of confirming transformation of the plant by a negative selection technique.

4. The method of claim 1, wherein the autoregulating promoter comprises P_{SAG12}.

5. The method of claim 1, wherein the ORF encodes a cytokinin.

6. The method of claim 5, wherein the ORF encodes the enzyme IPT (isopentenyl transferase).

7. The method of claim 1, wherein the autoregulating promoter is a plant-derived promoter selected from the group consisting of MAP kinase promoter, cyclin B gene promoter, CrRR1 promoter, maize opaque-2 promoter, rice phytochrome gene promoter, Arabidopsis ABI3 promoter, Arabidopsis ABI4 promoter, and Arabidopsis ABI5 promoter.

8. The method of claim 1, wherein the autoregulating promoter is a bacterial-derived promoter selected from the group consisting of luxC gene promoter, bvgp1 promoter, bvgp2 promoter, pasA promoter, pasB promoter, pasC promoter, and parD promoter.

9. The method of claim 1, wherein the autoregulating promoter is a mammalian promoter.

10. The method of claim 9, wherein the mammalian promoter is the TK promoter.

11. The method of claim 1, wherein the autoregulating promoter is a viral promoter.

12. The method of claim 11, wherein the viral promoter is the Cp promoter.

13. The method as set forth in any one of claims 7, 8, 9, 10, 11, or 12 in which the ORF is selected form the group consisting of CycD3, CBPs, CKI1, ARR, and GCR1.

14. The method of claim 1, wherein the transgene of interest is selected from the group consisting of raffinose synthase gene, polyunsaturated fatty acid desaturase genes, genes that confer to said plant resistance to nematode, fungi or other pathogens, and other economically important genes.

15. The method of claim 1, wherein the transgene of interest comprises bar.

16. The method of claim 3, wherein the negative selection technique comprises use of a glufosinate herbicide.

17. A transgenic plant produced using the method of claim 1, wherein said transgenic plant is a first generation transgenic plant or progeny thereof.

18. A method of transforming a plant with a transgene of interest, the method comprising the steps of:
(a) modifying a binary plant transformation vector for an Agrobacterium tumefaciens-mediated transformation to incorporate an expression cassette comprising a P_{SAG12} promoter coupled with an IPT coding region for control thereof and a transgene of interest to provide a modified binary vector for Agrobacterium tumifaciens; and
(b) using this modified binary vector for Agrobacterium tumifaciens to transform said plant.

19. The method of claim 18, wherein the plant is soybean.

20. The method of claim 18, wherein the transgene of interest is selected from the group consisting of raffinose synthase gene, polyunsaturated fatty acid desaturase genes, genes that confer to said plant resistance to nematode, fungi or other pathogens, and other economically important genes.

21. A transgenic plant generated using the method of claim 18, wherein said transgenic plant is a first generation transgenic plant or progeny thereof.

22. A method of transforming a plant with a transgene of interest, the method comprising the steps of:
(a) constructing a plant transformation vector that carries at least two T-DNA regions, wherein the first region contains the P_{SAG12}IPT cassette, the second T-DNA region carries a transgene of interest,
(b) incorporating the vector into an Agrobacterium; and
(c) using the Agrobacterium to transform a plant such that the P_{SAG12}IPT cassette is co-integrated into a T₀ plant genome with the transgene of interest.
(d) removing the IPT coding sequence from the plant genome of the progeny lines derived from the T₀ line.

23. The method of claim 22, wherein the plant is soybean.

24. The method of claim 22, wherein the transgene of interest is selected from the group consisting of raffinose synthase gene, polyunsaturated fatty acid desaturase genes, genes that confer to said plant resistance to nematode, fungi or other pathogens, and other economically important genes.

25. A transgenic plant generated using the method of claim 22, wherein said transgenic plant is a first generation transgenic plant or progeny thereof.
